(19) United States
(12) Patent Application Publication
(10) Pub. No.: US 2003/0036774 A1
(43) Pub. Date: Feb. 20, 2003

(54) Switching apparatus for monitoring catherter signals and performing cardioversion defibrillations

(76) Inventors: Chris Maier, Salt Lake City, UT (US); B. Tyler Clark, Draper, UT (US); Chrn Hwang, Provo, UT (US)

Correspondence Address: KIRTON AND MCCONKIE, 1800 EAGLE GATE TOWER, 60 EAST SOUTH TEMPLE, P O BOX 45120, SALT LAKE CITY, UT, 84145-0120, US

(21) Appl. No.: 09/870,156

(22) Filed: May 30, 2001

Related U.S. Application Data
(60) Provisional application No. 60/208,780, filed on Jun. 1, 2000.

(51) Int. Cl. A61N 1/39

(52) U.S. Cl. 607/5

(57) ABSTRACT

An apparatus for providing both a read/pace mode of operation and a cardioversion/defibrillation (C/D) mode of operation utilizing common electrodes is disclosed that switches between the various modes. The apparatus incorporates a switch, such an electromechanical or electronic switch, that is used to control any number of poles and allow all of the electrode connections to be switched simultaneously. The apparatus further includes a set of intracardiac catheters or coils, which are inserted into the body intravenously to monitor the heart and to provide C/D to the patient. A second set of electrical contacts or electrode patches can provide exterior operation on a patient and the apparatus can operate with sufficient current and intensity to operate in this mode, as opposed to the intracardiac mode for the catheter electrodes. The switch enables proper selection of either set of electrodes as well as power source and intensity to achieve optimal results in treatment and monitoring of a patient's heart.

### RELATED APPLICATION

The present application is related to and claims priority to U.S. Provisional Application Serial No. 60/208,780 filed Jun. 1, 2000, titled “Switching Box for Receiving Intracardiac Catheter Signals and Sending Cardioversion/Defibrillations,” which is incorporated by reference for all purposes.

### BACKGROUND

The present invention relates generally to monitoring and treating cardiovascular health. More particularly, the present invention relates to a cardiovascular treatment apparatus that is able to monitor cardiovascular activity as well as provide treatment stimulation using a common set of observation/treatment electrodes.

During electrophysiology studies (EPS), the majority of time is spent recording signals from intracardiac catheters. Certain EPS, however, require multiple cardioversion/defibrillations (C/D's). This is normally performed externally through two surface patches attached to the patient. This treatment also often causes residual discomfort to the patient in the form of skin burns and muscle aches due to the current applied to the patches and transferred through the patient's body. Performing internal C/D significantly reduces the amount of skin and muscle trauma to the patient.

There are other occasions that arise during the EPS and other cardiac emergencies that necessitate the use of a defibrillator to treat the cardiac arrhythmia. As previously mentioned, this has been done through external patches that are placed on the chest and another on the posterior side of the patient's thoracic region. In order to correct the patient's arrhythmia successfully using these external patches, a significant amount of energy is required to bring the heart back to a normal rhythm. This is due largely in part because the current must travel through body tissues that have high impedance, which tissues typically include bone, muscle, fat, and other tissues in order to reach the heart. A more effective and safer means of treating these arrhythmias is to apply the energy from within the heart. This can be done by delivering current from multiple catheters or coils strategically placed within the heart and surrounding areas. It can also be done using one intracardiac catheter and one external patch. In either case, the energy typically used in outside applications only is greatly reduced to treat the cardiac arrhythmia.

The benefits of using intracardiac electrodes to treat arrhytmia is that lower currents are possible, thereby causing less tissue damage during the current application. External cardioversion/defibrillation of the patient through the patches often leaves weeks of residual discomfort from these “shocks.” By performing the cardioversion/defibrillations from within the body, the tissues affected by the energy delivered are greatly reduced to a minimum and the patient rarely complains of discomfort.

In order to apply enerty to intracardiac catheters or coils, one must utilize a means to deliver the energy from the external debrillator to those intravenously placed catheters. This is currently done by removing the cable connectors from the monitoring system and plugging them into a device that is linked to the defibrillators positive and negative poles. Once this is done, one can deliver the designated energy levels for treatment. During this time, there are no intracardiac electrograms being recorded. After the current is delivered, the the intracardiac catheter interface cables must be removed from the connection device and plugged back into the monitoring input modules. This causes another problem and that is of unplugging and plugging catheters in and out for either treatment or observation. This is a time-consuming operation since both catheters have multiple pins that need to be plugged in independently and in correct order. During this time of switching from the C/D output interface to the recording box, no electrograms can be recorded.

Accordingly, what is needed is an apparatus that is able to perform both electrogram monitoring and cardioversion/defibrillation events using the same electrodes, but without requiring removal from a first device to be attached to a second device and vice versa in order to have both operations utilize the same set of electrodes.

### SUMMARY OF THE INVENTION

According to the present invention, an apparatus for providing both a read/pace mode of operation and a cardioversion/defibrillation (C/D) mode of operation utilizing common electrodes is disclosed that switches between the various modes. The apparatus incorporates a switch, such an electromechanical or electronic switch that is used to control any number of poles and allow all of the electrode connections to be switched simultaneously. The apparatus further includes a set of intracardiac catheters or coils, which are inserted into the body intravenously to monitor the heart. The catheters include metal rings to assist in carrying out the defibrillation charge as well. By switching from positive to negative, a current is created that travels through electrical cables connecting the catheters to the treatment and monitoring devices.

The apparatus operates in one of two modes. The first mode is a receiving mode where the apparatus receives signals from the chart through the catheter to the apparatus. This receiving mode then also records the information, amplifies it, and forwards it to a monitor for viewing by the care-giver.

The second mode of operation is a shock mode, which shunts off the information channels forwarded to the monitor and recorder and instead provides an electrical current through the cables to the catheter and the several electrodes located on the catheter to treat the heart. The utilization of positive and negative signals provides a shock to the heart for defibrillation. The apparatus can then be switched back to the monitoring or receive mode, which monitors the heart activity using the same catheter electrodes.

The apparatus can incorporate the defibrillating treatment device with the switch and the electrophysiology monitoring device in an alternative embodiment. Further still, the apparatus can comprise a treatment and monitoring device that is wirelessly coupled to a control device or base. Signals are sent between the two devices to provide remote monitoring and treatment of a patient. This embodiment eliminates the problems of electromagnetic field interference (EFI) and radiation field interference (RFI) that normally occur when both signal processing and signal detection elements are embodied in the same system proximate one another. Further still, the system enables exterior patches to be attached to the patient and connected to the apparatus as an alternative to the internal electrodes. In this embodiment, the patches receive a greater current than would normally be applied to the intracardial electrodes. Further still, isolation circuitry is added to prevent application of too great a current when operating in the monitoring and treatment mode.

### BRIEF DESCRIPTION OF THE DRAWINGS

The foregoing and other objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described and explained with additional specificity and detail through the use of the accompanying drawings in which:

FIG. 1 illustrates a schematic diagram of the electrophysiology apparatus and switch in accordance to the present invention;

FIG. 2 illustrates a schematic diagram of the switch of FIG. 1, in accordance to the present invention;

FIG. 3 illustrates an interior view of a patient's heart wherein the electrodes are placed intra-cardially;

FIG. 4 illustrates an exterior view of a patient wherein electrode patches are placed on the skin surface in an alternative embodiment;

FIG. 5 illustrates the same exterior view of FIG. 4, but includes a posterior view of the patient and different electrode placement; and,

FIG. 6 illustrates a block diagram of a treatment and monitoring apparatus that operates in a wireless mode.

### DETAILED DESCRIPTION OF SPECIFIC EMBODIMENTS

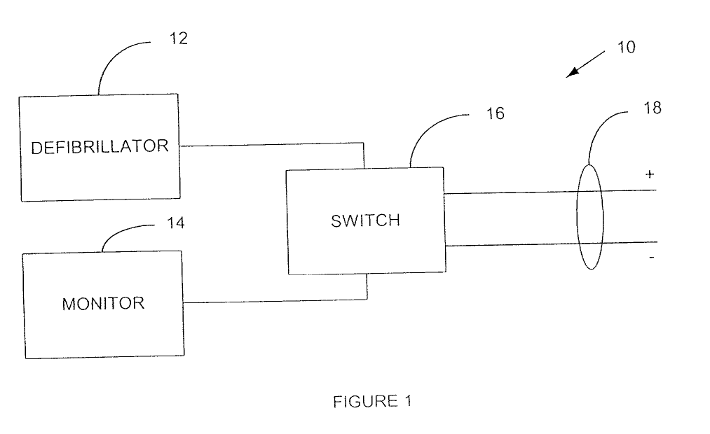

It will be readily understood that the components of the present invention, as generally described and illustrated in the figures herein, could be arranged and designed in a wide variety of different configurations. Thus, the following more detailed description of the embodiments of the system and method of the present invention, and represented in FIGS. 1 through 6, is not intended to limit the scope of the invention, as claimed, but is merely representative of embodiments of the invention.

The specific embodiments of the invention will be best understood by reference to the drawings, wherein like parts are designated by like numerals throughout.

FIG. 1 illustrates a monitoring and treatment apparatus 10 in accordance with the present invention. Apparatus 10 includes a cardiac-treatment device or defibrillator 12 and a monitoring device 14. A switch 16 is provided to enable a set of electrodes 18 to be selectively connected to either monitoring device 14 or defibrillator 12. Electrodes 18 further include a connecting pin at one end to provide electrical connection to apparatus 10 and a catheter at the other end to insert within a patient for monitoring and treatment intracardiacally. Each catheter includes a plurality of conductive surfaces to perform both monitoring and treatment functions. Two catheters are utilized for performing internal cardioverion/defibrillation (C/D). One catheter or electrode 18 is placed in the right atrium and the other electrode 18 is placed within the coronary sinus. The electrode catheters can be utilized for multiple types of cardiac arrhythmias that may require that the catheters be placed in different areas of the heart. Thus, the system is capable of performing atrial and ventricular arrythmias treatments, which eliminates the need to change connections, reducing treatment time and improving monitoring ability.

Apparatus 10 can be implemented in different embodiments. One embodiment contemplated has separate devices that are coupled together via a discrete switch 16. Defibrillator 12 and monitoring device 14 are standard devices known to those skilled in the art and operate independently of one another. Switch 16 is utilized to connect electrodes 18 selectively to either device and to provide isolation from the two devices as the current generated by defibrillator 12 for treatment of a patient (not shown) could damage monitoring device 14. In an alternative embodiment, apparatus 10 integrates defibrillator 12 with monitoring device 14 and switch 16. Thus, no cable connectors are needed to connect switch 16 to either defibrillator 12 or monitoring device 14. A hard wiring of each device through switch 16 would then be provided. Further, apparatus 10 can be made in a portable model with its own power supply, such as rechargeable batteries or the like.

Defibillator 12 provides electrical current through electrodes 18 to stimulate a patient's heart intracardiacally. Defibrillator 12 delivers a current to the heart from 1 to 360 joules, with a more defined range of 3-10joules while performing internal C/D's. This defined range is sufficient to perform the desired defibrillation necessary or the cardioversion. Defibrillator 12 includes the necessary circuitry to convert current from a convention source, such as from a wall socket or batteries, to the controlled current range along with sufficient safety circuitry to prevent an over stimulation from occurring. Significantly, a desired energy level is applied to the heart for treatment. This energy is in the form of a selected voltage and selected current to match the impedance of the patient's heart or body tissue to provide proper and responsive treatment.

Monitoring device 14 performs the functions of monitoring the electrical signals generated by the patient's heart during electrophysiology studies. The electrical signals are amplified and converted to useful information for display to the care giver either in video or printed output. Monitoring device 14 can also record the signals received and converted for later review. Monitoring device 14 includes the appropriate electronics to perform signal sensing, amplification, display, recording, and printing as necessary. Further, it is desired that there be isolation of the electrical current from passing through the switch during read mode, which could adversely affect the quality of the signals. Thus, the energy source is placed outside or isolated from monitoring device 14 either via an external transformer or through signal attenuation or blocking filters, such as capacitors and resistors, or their equivalents. During treatment mode, since no signal is to be monitored, it is not necessary to continue isolation, except of course to prevent a damaging shock from feeding back to the monitoring equipment through the electrodes.

Each device 12 and 14 would normally require independent contacts on the patient or would necessitate swapping the leads from one device to the other. Swapping leads is time consuming and inefficient. The contacts for the leads typically require more than one contact for each patch or catheter. With multiple catheters and patches being utilized, the number of contacts needed to be switched grows geometrically. It is the use of common patches or catheters, or both, and switching between them using electronic means, instead of manual or even simple mechanical switching, that overcomes the problem of the prior art. Further, with the cost of the monitoring devices and the treatment devices being very expensive, it is advantages to be able to connect discrete monitoring elements together. Further, the switching scheme disclosed within the present invention can also be integrated into a single system that allows for switching to be done internally, without even needing to hook up individual monitors or treatment devices. This allows monitoring with one set of probes to be done and to provide treatment, when necessary, with the same probe set.

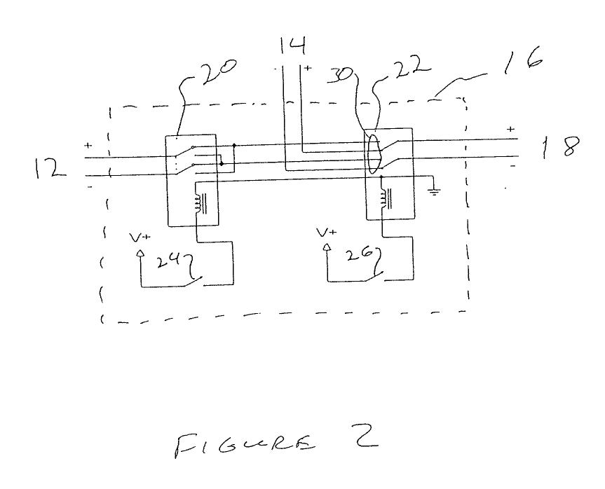

Switch 16 is shown in greater detail in the schematic diagram of FIG. 2. In the discrete embodiment, switch 16 includes a direct current input, such as from a wall socket power source or from portable batteries, that is used to supply energy to internal relays and other electronics not shown. In the integrated embodiment, switch 16 derives its power from a universal power source used to power defibrillator 12 and monitoring device 14. In either case, input energy is routed through an on/off switch so that overall power of apparatus 10 or switch 16 can be controlled. In the discrete embodiment, switch 16 incorporates at least two sets of multiple pin jack connections, which offer different series of connections to provide a positive and a negative lead. A first series of pin jack connectors connects electrodes 18 with defibrillator 12 while a second series of pin jack connectors connect electrodes 18 with monitoring device 14. Each series of pin jack connectors further connect to a relay switch 22. Defibrillator 12 is further coupled to a second switch 20, which will be described in greater detail below.

Relay switch 22 is a double throw relay having a normally closed (NC) relay terminal 30, which is connected to both monitoring device 14 and defibrillator 12. This is the standard configuration for switch 16 and provides connection of the electrodes 18 with monitoring device 16. Relay switch 22 further connects to a power supply V+via control switch 26. When control switch 26 is closed, energy is applied to relay 22, which then connects electrodes 18 with defibrillator 12. This configuration represents the cardioversion/defibrllation treatment mode.

A single switch can be utilized to control all the relays within the apparatus. For example, each catheter has at least two leads. The switch can switch from a read or monitor position to a shock or treatment position. The switch directs a current to each of the relays within the apparatus when placed in treatment mode, causing the relays to switch to the treatment mode. Once the treatment is completed, the switch returns to the monitor position and isolates the relays from current normally needed for the treatment mode.

A polarity switch 24 couples a energy V+with relay switch 20. When polarity switch 24 is closed, energy is supplied to relay 24, which is a double pole/double throw (DPDT) relay. Relay 24 is configured such that when voltage is applied, its contact closure changes the input from defibrillator 12 such that the polarity of the voltage supplied to each series of pin jack connections is reversed. Polarity switch can be opened and closed rapidly in an automated fashion to reverse the polarity and generate an alternating current to shock the patient's heart.

When control switch 26 is open, apparatus 10 is in monitoring mode and monitoring device 14 receives signals that travel via electrodes 18. The electrical signals received from the patient are amplified and displayed for the caregiver. In this mode, defibrillator 12 is isolated from electrodes 18 and from monitoring device 14. It should be noted that each of the switches can be integrated within a common omnibus switch unit, which has electrical switches to effect the desired functions previously mentioned. For example, polarity switching and control switching can be performed within the same overall unit.

When apparatus 10 is switched to treatment mode, no information is forwarded to the monitor and no information is recorded. Rather, an electrical current is transmitted from defibrillator 12 through electrodes 18 to the electrical contacts located on the catheter as placed within the patient's heart. The use of a positive and negative signal provides a shock to the heart, which serves to defibrillate the heart. Apparatus 10 then can be switched back to the read mode of operation utilizing the same electrodes to monitor the heart condition. Defibrillator 12 generates from between 3 to 30 joules of current for performing defibrillation. This low current amount is possible since the current is applied directly to the heart instead of applied externally through the chest. This also eliminates or avoids the problems of arcing on the skin, muscle aches from electrical shock, and muscle damage from the electrical shock.

Switch 16 can be implemented using electromechanical switches or it can be implemented in a fully electronic embodiment, or a combination thereof. For example, polarity relay 20 can incorporate power transistors and switching transistors to control current from defibrillator 12 to electrodes 18. Relay 22 also can incorporate similar power transistors and switching transistors to control the delivery of current from defibrillator 12 to electrodes 18 and of current from the patient's heart transmitted via electrodes 18 to monitoring device 16. It is understood that additional circuitry would be necessary, including capacitors, transformers, amplifying transistors, and resistors, which is left to the skilled artisan for implementation and design.

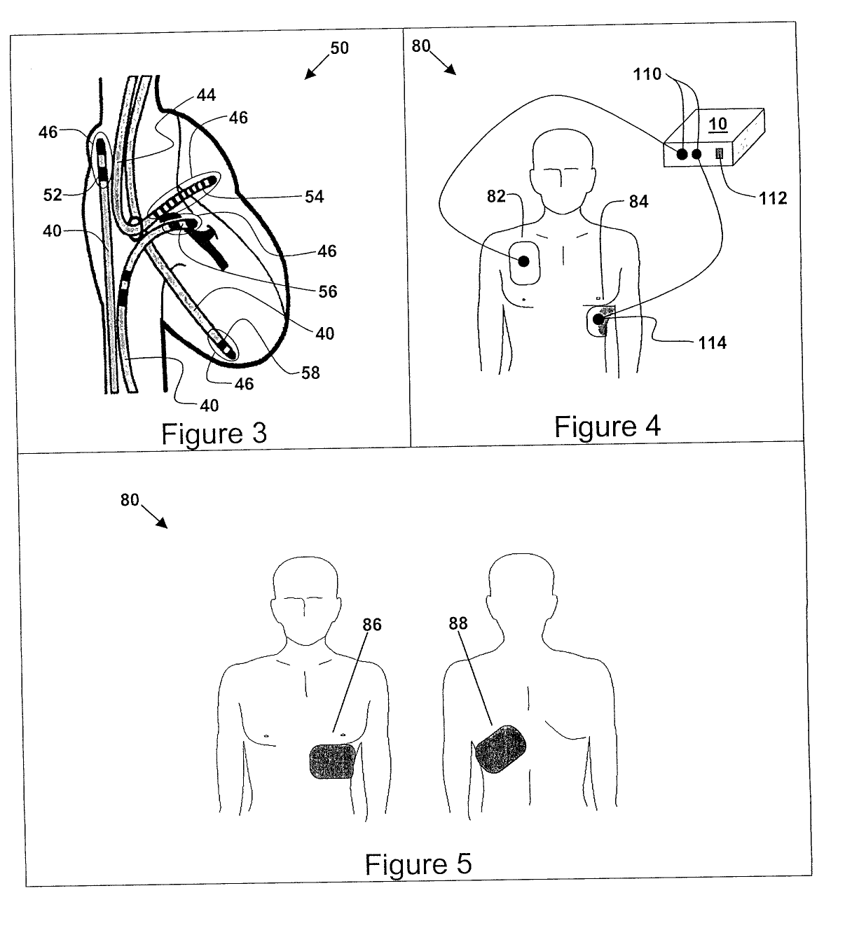

Electrodes 18 are shown in greater detail in the diagram of FIG. 3. Electrodes 18 are placed intracardiacally within a patient's heart 50. Each electrode comprises a conducting wire 40, which is utilized to connect a connector pin with a catheter 44. A connector pin connects electrode 18 with switch 16, as previously shown in FIG. 1. Each catheter 44 is intravenously inserted in the patient so as to reach the patient's heart. Catheter 44 includes a plurality of electrical contacts 46, which improve the surface contact area intracardiacally to provide greater monitoring performance and electrocardiac stimulation. Contacts 46 are made from an inert and physiologically compatible material, such as surgical stainless steel or platinum.

Electrodes 18 are positioned as shown within heart 50 so that contact is made within the high right atrium 52 location, within the coronary sinus 54, within the his bundle 56, and within the right ventricle 58. These locations serve to provide both stimulation when needed as well as monitoring of heart 50. Thus, in this embodiment, two catheters are utilized for the internal cardioverion/defibrillation (C/D) process. One is placed in the right atrium and the other in the coronary sinus. Alternatively, the apparatus can be utilize to treat multiple types of cardiac arrhythmias that may require that the catheters be placed in different areas of the heart.

In another alternative embodiment, which is illustrated in FIG. 4, apparatus 10 has been modified to include a pair of output jacks 110. Corresponding switches 112 are provided in apparatus 10 to allow a set of external electrode patches 114 to connect to the skin of the patient 80, such as on the right sternum 82 and on the left apex 84, forming an anterolateral placement and plugged into apparatus 10 via output jacks 110. Patches 114 provide external defibrillation in the event that the internal C/D operation fails. Switches 112 are switched to form a current path between the external patches rather than through the internal electrodes. This allows the defibrillator apparatus 10 to remain electrically switchably connected to both the internal electrodes and to the external patches thereby providing the ability to perform both internal or external C/D, as necessary. It is imperative to terminate ventricular arrhythmias as soon as they start. Thus, prior to adding these two switches to the apparatus it was necessary for a second defibrillator to be connected to the patients external patches. This increases the cost of the necessary equipment required to do an Electrophysiology study. This addition to the device now allows for a single defibrillator to be used and allows for immediate selection of internal or external C/D. Of course, the defibrillator generates currents suitable for both internal use and external use, the external use current being great enough to penetrate through the patient's skin, muscle, tissue, and chest region. The internal current is the same as that previously described, which is much lower than the external current level since the treatment internally is directly to the heart muscle and not through dense body tissue or bone.

FIG. 5 illustrates the same patient 80 of FIG. 4, but with the exterior pads placed one on the patient's posterior 88 and the second on the patient's anterior 86, both adjacent the patient's heart.

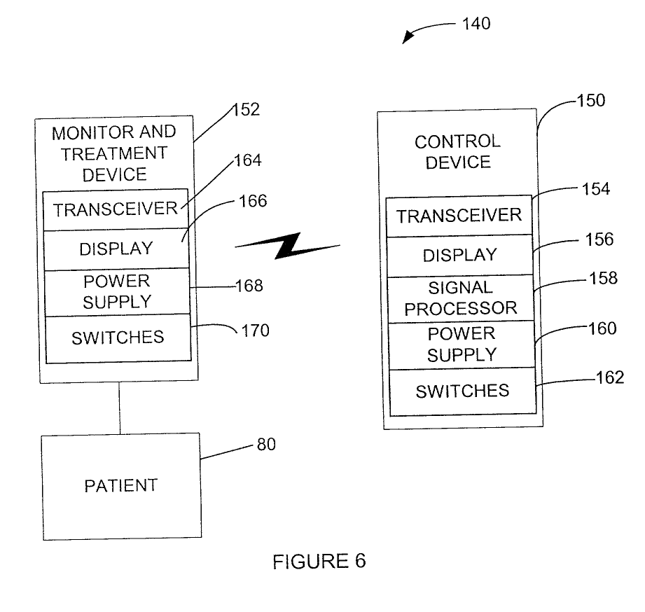

In yet another alternative embodiment, which is shown in detail in FIG. 6, a wireless apparatus 140 can include a remote control device 150, which is in wireless communication with a monitor and treatment device 152, to control the mode of operation between monitoring and treatment. Control device 150 includes a transceiver 154, which operates as both a transmitter and receiver, a display 156, a signal processor 158, a power supply 160, and a switch 162.

Treatment device 152 also includes a similar transceiver 164 to send and receive wireless signals from control device 150. Treatment device 152 utilizes electrodes 18 either as internal contacts for intracardiac treatment and monitoring, or external electrodes, such as shown in FIGS. 4 and 5, for external treatment and monitoring, or both. Treatment device 152 operates to perform both treatment and monitoring of a patient. Switches 170 provide for the ability to switch between treatment and monitoring modes of operation as provided in apparatus 10 described above. A power supply 168, such as batteries or a power cord with proper power management elements, provides power for both monitoring functions and electro-shock treatment. A display 166 is provided that shows operation information such as whether the device is in monitor or operating modes as well as to display whether a malfunction of the system has occurred or urgent attention is needed, or the batteries are getting low.

During monitoring mode, treatment device 152 transmits signals from the monitoring operation back to control device 150. Control device 150 processes the signals via processor 158 for display on display 156. Switches 162 enable the medical practitioner to control which mode of operation device 152 is performing, whether it be treatment or monitoring. The practitioner can also view the patient's statistics, as displayed on display 156 and select treatment if warranted.

Display 156 enables the user to view the monitored operation of apparatus 10, such that the user can view the patient's vital signs remotely. If, based on the information displayed on display 156, the user needed to provide treatment to the patient, such as the patient went into cardiac arrest, the switch 170 can be activated by the user via switch 162, to send a signal to device 152 to perform defibrillation.

Treatment device 152 would have a limited power source and would transmit the monitor signals to a control unit 150, which would then process the signals for display on a monitor. The desirability of this embodiment is that the processing circuitry used to process the signals and the power supply circuitry can interfere with the signal either in electromagnetic interference or radio-frequency interference, so isolating the monitoring apparatus from the processing apparatus and its main power supply avoids these interference problems.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed is:

1. An electrophysiology apparatus comprising:
a defibrillator to provide electrical stimulation for cardioversion or defibrillation, or both;
a electrophysiology monitoring device to monitor electrocariograms/cardiophysiology during an electrophysiology event;
a plurality of electrodes, each having a first and second end, the first end being intracardiacally inserted for operation; and
a switch to selectively couple the second end of each electrode either to the defibrillator or to the monitoring device.

2. The apparatus of claim 1, wherein the switch further comprises a double pole double throw (DPDT) relay to selectively couple the set of electrodes either to the defibrillator or to the monitoring device.

3. The apparatus of claim 1, wherein the switch further comprises a polarity switch to alternate the polarity of the electrodes as coupled to the defibrillator.

4. The apparatus of claim 1, wherein each electrode includes a pin connector on one end to connect to the switch and a catheter on the other end, which is electrically connected to the pin connector.

5. The apparatus of claim 2, wherein the switch further comprises a control switch to couple a energy source to the DPDT relay thereby switching connection of the electrodes from the monitoring device to the defibrillator.

6. The apparatus of claim 1, further comprising a second set of electrodes, each having a first end and a second end, the first end being placed on an exterior surface of a patient and the second end coupled to the switch, such that the switch selectively enables the second set of electrodes to connect to the defibrillator and the monitoring device exclusive of the plurality of electrodes.

7. The apparatus of claim 1, further comprising a remote switch, wirelessly coupled to the switch, to enable remote operation of the apparatus.

8. A switching apparatus for connecting a set of electrodes alternatively to a defibrillator or to an electro-physiology monitoring device, comprising:
a set of electrode contacts;
a set of monitoring output contacts;
a set of defibrillator input contacts; and
a double pole, double throw (DPDT) relay, coupled to the set of electrode contacts, the set of monitoring output contacts, and the set of defibrillator input contacts, wherein a normally closed (NC) position of the DPDT relay couples the set of electrode contacts with the set of monitoring output contacts and an active position couples the set of electrode contacts with the set of defibrillator input contacts.

9. The switching apparatus of claim 8, further comprising a polarity switch, coupled between the set of defibrillator input contacts and the DPDT relay, to switch polarity of the set of contact electrodes when the DPDT relay is in the active position.

10. The switching apparatus of claim 8, further comprising a second set of electrode contacts, each having a first end and a second end, the first end being placed on an exterior surface of a patient and the second end coupled to the switch, such that the switch selectively enables the second set of electrodes to connect to the defibrillator input contacts and the monitor output contacts exclusive of the plurality of electrode contacts.

11. The switching apparatus of claim 8, further comprising a transceiver, coupled to the DPDT relay.

12. An electrophysiology apparatus comprising:
a control device, having a transceiver, a signal processor, a power supply, and a switch; and,
a monitoring and treatment device, comprising:
a transceiver, for transmitting and receiving signals from the control device;
a power supply, coupled to the transceiver;
a defibrillator, coupled to the transceiver and the defibrillator, to provide electrical stimulation for cardioversion or defibrillation, or both;
a electrophysiology monitoring device, coupled to the transceiver and the power supply, to monitor cardiophysiology during an electrophysiology event;
a plurality of electrodes, each having a first and second end, the first end being intracardially inserted for operation; and
a switch to selectively couple the second end of each electrode either to the defibrillator or to the monitoring device;
wherein signals are transmitted between the monitoring and treatment device and the control device, which signals are processed by the signal processor to represent the health state of a patient being monitored or are used to control the monitoring and treatment device.

13. The apparatus of claim 12, wherein the switch further comprises a double pole double throw (DPDT) relay to selectively couple the set of electrodes either to the defibrillator or to the monitoring device.

14. The apparatus of claim 12, wherein the switch further comprises a polarity switch to alternate the polarity of the electrodes as coupled to the defibrillator.

15. The apparatus of claim 12, wherein each electrode includes a pin connector on one end to connect to the switch and a catheter on the other end, which is electrically connected to the pin connector.

16. The apparatus of claim 13, wherein the switch further comprises a control switch to couple a energy source to the DPDT relay thereby switching connection of the electrodes from the monitoring device to the defibrillator.

17. The apparatus of claim 12, wherein the monitoring and treatment device further comprises a second set of electrodes, each having a first end and a second end, the first end being placed on an exterior surface of a patient and the second end coupled to the switch, such that the switch selectively enables the second set of electrodes to connect to the defibrillator and the monitoring device exclusive of the plurality of electrodes.

18. The apparatus according to claim 12, wherein the control device further comprises a display, coupled to the signal processor.

19. The apparatus according to claim 12, wherein the control device can transmit signals to the monitoring and treatment device to select between monitoring or treatment operations via the control device switch.

20. The apparatus according to claim 12, wherein the monitoring and treatment device further comprises a display, coupled to the electrodes.
